(19) United States
(12) Patent Application Publication
(10) Pub. No.: US 2023/0211153 A1
(43) Pub. Date: Jul. 6, 2023

(54) ELECTRICALLY CONDUCTIVE CONNECTION ELEMENT FOR A TEMPORARY ELECTRICALLY CONDUCTIVE CONNECTION TO AN ELECTRICAL CONSUMER

(71) Applicants: Fraunhofer-Gesellschan zur Férderungder Angewandten Forschung E.V., München (DE); Technische Universität Dresden, Dresden (DE)

(72) Inventors: Peter QUADBECK, Dresden (DE); Georg PÖHLE, Dresden (DE); Christian REDLICH, Dresden (DE); Utz KAPPERT, Dresden (DE)

(21) Appl. No.: 18/000,518

(22) PCT Filed: Jun. 2, 2021
(86) PCT No.: PCT/EP2021/064818

§ 371 Date: Dec. 2, 2022

(30) Foreign Application Priority Data
Jun. 5, 2020 (DE) 10 2020 207 093.8

(51) Int. Cl. A61N 1/05

(52) U.S. Cl. CPC A61N 1/056; A61N 1/0595

(57) ABSTRACT

An electrically conductive connection element for a temporary electrically conductive connection to an electrical consumer, in particular an external pacemaker, which is implanted in the living tissue of a patient, may have at least one biocompatible and bioresorbable electrical conductor strand in the form of an individual wire or a multifilament formed by multiple wires. The at least one electrical conductor strand is surrounded by at least one biocompatible and bioresorbable polymer or is embedded in a matrix formed by the polymer.

The invention relates to an electrically conductive connection element consisting of a line and an electrode region for a temporary electrically conductive connection to an electrical consumer that is in particular an external pacemaker. The electrically conductive connection element can be implanted in the living tissue of a patient.

Such electrically conductive connection elements are routinely temporarily anchored in the atrial or ventricular myocardial tissue following surgical procedures on the heart. Said connection elements are in most cases used for 4 -7 days and for a maximum of up to some weeks after the surgery. The connection element is pulled out and disposed of after satisfying its function. The metallic core of the line in accordance with the prior art consists of corrosion resistant stainless steel (WNr. 1.4404/AISI 316L). The steel wire is sheathed with a biocompatible polymer (e.g. polyethylene (PE) or polyethylene terephthalate (PET)) for electrical insulation toward the surrounding tissue. The total diameter of the electrical line is generally between 0.2 mm and 0.4 mm. In a number of cases, the connection element cannot be pulled or can only be incompletely pulled due to growths in the tissue and is then cut off close to the skin surface. The remainder permanently remains in the organism as a foreign body. This carries the risk of a long term inflammation reaction or foreign body reaction. There is moreover the risk of a migration of the remainder within the thorax and abdomen. Serious complications such as arrhythmias, pericardial tamponades, bleeding, or infections can occur in rare cases as a result of the pulling out of the connection element. The described long term or acute complications result in additional strains for the patient as well as in costs and effort.

It is therefore the object of the invention “Electrically conductive connection element for a temporary electrically conductive connection to an electrical consumer” to provide electrically conductive connections between an electrical consumer and the body tissue of the patient, in particular between an external pacemaker and the heart tissue of the patient, that degrade and are resorbed in the body within a short time after the end of the operation of the electrical consumer so that the removal or the permanent remaining in the body can be avoided.

This object is achieved in accordance with the invention by electrically conductive connection elements having the features of claim 1. Advantageous embodiments and further developments of the invention can be implemented using features designated in subordinate claims.

In the sense of the invention, a material is called bioresorbable when it can degrade in the body and the degradation products are either directly eliminated from the body or are used as part of the regular metabolic processes or are converted into forms that can be utilized by the body. An implant or a part of an implant that is formed from a bioresorbable material loses its original shape over time after implanting. The concentrations of the elements contained in the material in the body can exceed the normal values during degradation. These concentrations return to the normal values after the complete degradation of the material at the site of the implanting.

An electrically conductive connection element for a temporary electrically conductive connection to an electrical consumer, in particular to an external pacemaker, can in particular be implanted in the living tissue of a patient. It consists of at least one biocompatible and bioresorbable electrical conductor strand in the form of a single wire or of a multifilament formed from a plurality of wires.

The electrically conductive connection element can be formed with at least one conductor strand that consists of the bioresorbable materials molybdenum (Mo), tungsten (W) or a Mo or W base alloy.

In this respect, a Mo base alloy consists of at least 50 at% Mo and can be formed from at least one of the alloy elements selected from W, Re, V, Nb, Ta, Hf, Mn and Fe. A W base alloy consists of at least 50 at% W and can be formed by at least one of the alloy elements selected from Mo, Re, V, Nb, Ta, Hf, Mn and Fe. A conductor strand can be a single wire or can be a multifilament formed from a plurality of wires.

The at least one conductor strand is surrounded by or embedded in at least one biocompatible and bioresorbable polymer. In preferred embodiments of the invention, it is one or more polymers selected from polylactide, polygly-colide, poly(lactide-co-glycolide) and polycaprolactone. Copolymers and mixtures of said polymers can furthermore be used. These polymers are those that are resorbed in the body over a reasonable time period, for example within a few months. An electrical insulation toward the surrounding body tissue is achieved by the sheathing or embedding of the at least one conductor strand. The invention in particular comprises the use of a plurality of polymers for the formation of a sheath of the conductor strands in order in this manner to achieve an advantageous combination of electrical insulation during the period of use and fast degradation in the body after the end of the operation of the electrical consumer.

The essence of the invention therefore consists of a temporary electrical connection line, in particular for external pacemakers, that is formed with at least one conductor strand of bioresorbable meal and an electrically insulating sheath of at least one bioresorbable polymer.

Mo and W are particularly suitable as materials for bioresorbable electrical conductor strands. These metals dissolve into ions, that are renally eliminated, under physiological conditions due to steady surface corrosion. The degradation behavior is thus substantially more easily predictable with respect to other known bioresorbable metals, such as magnesium, that tend to localized corrosion. In addition, the oxide layers that may form on the surface are likewise bioresorbable, which represents an advantage over iron that forms oxide layers that degrade very poorly under physiological conditions. Mo and W additionally combine a low specificity electrical resistance (in each case approximately p = 0.05 Ω•mm²/m) with high mechanical strength (stress-relief annealed Mo: Rₑ ≈ 600 MPa, tensile strength Rₘ ≈ 700 MPa; W: typically Rₑ > 500 MPa, Rₘ > 900 MPa). The specific electrical resistance, that is lower by a factor of 15 in comparison with 316 L (p = 0.75 Ω•mm²/m) permits a substantial reduction of the conductor cross-sectional area with the same electrical resistance. The high mechanical strength permits a production and processing of flexible and mechanically stable wires and also a use as conductor strands within the described connection element without any breakage occurring.

The ends of the mechanical wire or wires used for the conductor strands can be machined so that they can take over the function of a surgical needle, an electrode, a connection to a conventional surgical needle, or a connection to an external electrical consumer.

The time period up to the complete resorption can be influenced by the design of the conductor strands. An embodiment having a large number of very thin individual wires or multifilaments of a plurality of very thin wires reduces the time period required for the complete resorption by a large exposed metal surface. The diameter of the individual wires can here be selected in the range between 3 µm and 50 µm, preferably in the range between 10 µm and 30 µm.

Said polymers can form a previously not described temporary electrical insulation of the bioresorbable electrical conductor strands in combination with said metal wires.

Preferred embodiments of the invention will be described in the following.

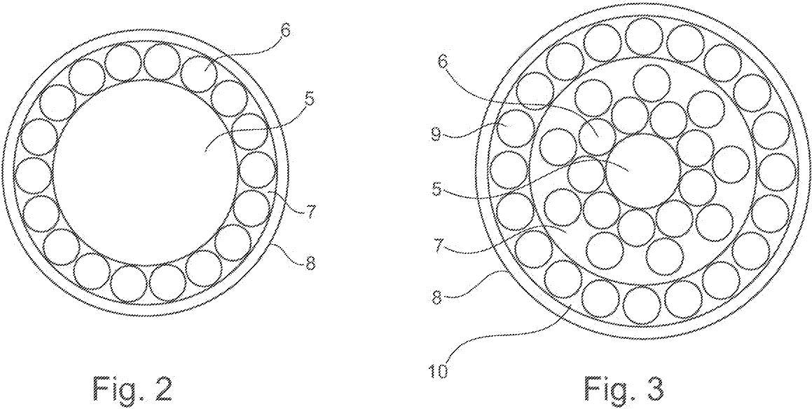

In a possible embodiment in accordance with FIG. 2, metallic conductor strands are wound around a core that is formed from a polymer A degradable in a short time. The conductor strands are embedded in a first inner layer. The first inner layer consists of the bioresorbable polymer A from which the core is also formed. The first inner layer is completely surrounded by an outer layer that is formed from a polymer B that is degradable more slowly in comparison with polymer A or from a polymer B that has a delayed start of the degradation in comparison with polymer A. The outer layer of polymer B is designed such that the conductor strands are electrically insulated toward the surrounding tissue for the intended time period of the connection to an external consumer of a few days up to some weeks. The outer layer of polymer B is subsequently degraded and exposes the first inner layer. The first inner layer, the conductor strands, and the core dissolve in the body within a short time due to the higher degradation rate of polymer A and due to the design of the conductor strands as very thin wires.

In a further preferred embodiment shown schematically in FIG. 3, further conductor strands can be wound on the outer surface of the first inner layer. A second inner layer from the same polymer A is additionally formed. The second inner layer is completely surrounded by the outer layer of the more slowly degradable polymer B. A two-conductor connection element can thus be provided, with the two groups of conductor strands having different functions being electrically insulated from one another over a sufficiently long time period. In this sense, embodiments having more than two inner layers that surround metallic conductor strands are also possible. Embodiments are equally possible in which the layers that surround metallic conductor strands are separated from one another by layers of the more slowly degradable polymer B. Embodiments are equally possible in which further polymers are used that have degradation behaviors differing from the polymers A and B and that can be resorbed in time periods of different lengths.

In a further preferred embodiment, at least two electrical conductor strands having different functions can be arranged coaxially or coradially with one another in the at least one inner layer formed with polymer A. The electrical conductor strands embedded in the different layers can satisfy different functions in this manner. They can e.g. be used as anode and cathode lines for an embodiment as bipolar electrical connection lines and as electrical lines for separate measurement, stimulation, and defibrillation electrodes.

In a preferred embodiment, the polymers A and B are built up of the same monomer, e.g. lactide, and have different molecular masses, e.g. 10,000 Da for polymer A and 20,000 Da for polymer B. In this respect, a higher molecular mass causes a more slowly running hydrolysis and degradation process of the polymer. Longer chains of monomers, a greater branching of the chains, and a greater crosslinking of the polymer chains are inter alia the cause of this. In a further preferred embodiment, polymer B is a homopolymer, e.g. polylactide, and polymer A is a copolymer, e.g. poly(lactide-co-glycolide) having the respective same molecular mass of e.g. 10,000 Da. In this case, the degradation rate of poly(lactide-co-glycolide) is greater than that of the pure polylactide. In a further preferred embodiment, polymers A and B are copolymers or blends of the same monomers while the crystallinity or order of the polymer chains differs. In this case, polymer A having a smaller crystallinity is degraded faster than polymer B having a higher crystallinity.

The polymer forming the outer surface should in particular provide insulation of the at least one electrical conductor strand from direct or indirect electrical contact with the body tissue and prevent a premature oxidation of the at least one conductor strand for a time period of preferably at least 14 days.

In this sense, a layer of polymer B should electrically insulate the at least one conductor strand at least during the functional time period of typically fewer than 7 days. Polymer B should preferably electrically insulate the at least one conductor strand for at least 14 days with a safety factor of 2. Polymer A, in contrast, can act only as a stabilizing substrate for the electrical conductor strands for this purpose and should dissolve after the end of the required functional time period in a short time where possible.

In an embodiment of the invention, the polymer-free region is modified as an electrode region that is in direct contact with the heart tissue in such a way that a change of the electrical properties of the polymer-free electrode region is prevented over the functional time period, i.e. during the regulation or monitoring of the heart action of the patient by an external pacemaker. An unhindered transmission of the current pulses emanating from the external pacemaker is thereby ensured. This is achieved by a modification of either only the polymer-free electrode region or of the total conductor strands of the connection conductor.

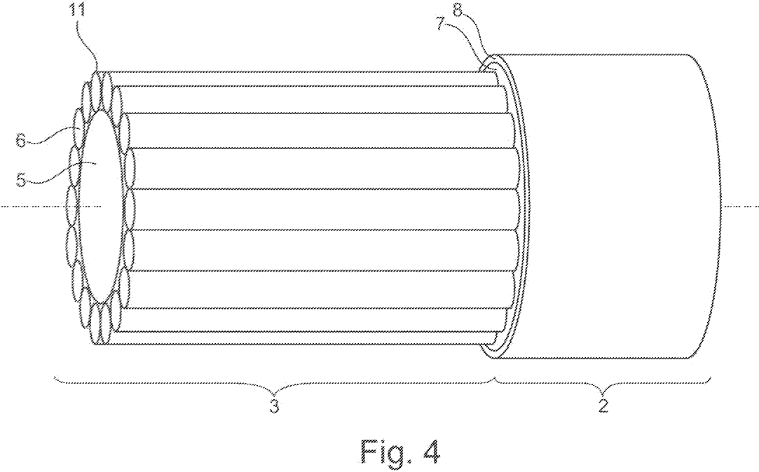

It is also possible to use a connection element having a modified polymer-free electrode region in the invention, as is indicated by FIG. 4. In comparison with an example such as is shown in FIG. 2, a coating of the conductor strands has been carried out that is formed from a biocompatible, likewise resorbable and in this respect less oxidation sensitive metallic connection on a Mo or W base than that of the conductor strands themselves. Possible application methods are inter alia a galvanic coating, chemical vapor deposition, plasma coating, and diffusion annealing. The coating can also be formed from resorbable, conductive oxides, nitrides, or carbides of the material of the conductor strands. These oxides, nitrides, or carbides can e.g. be obtained by a heat treatment of the electrical conductor strands by high temperature oxidation, nitration, or carbonization in an atmosphere rich in oxygen, nitrogen, or carbon.

The main advantage of this invention over the prior art thus consists of residues of the electrically conductive connection to a temporary pacemaker remaining in the body of a patient being completely and predictably degraded and resorbed within a defined time period after satisfying its function. Acute and long term complications due to both the remaining and to the pulling out of temporary connection lines can thereby be avoided. The invention is primarily provided for use as an electrically conductive connection to an external pacemaker. It can, however, be used in all cases in which electrical lines having only temporary functions have to be implanted in a patient.

The invention will be explained in more detail in the following with reference to examples. In this respect, the individual features can be combined with one another independently of the respective example or of a figurative representation.

There are shown:

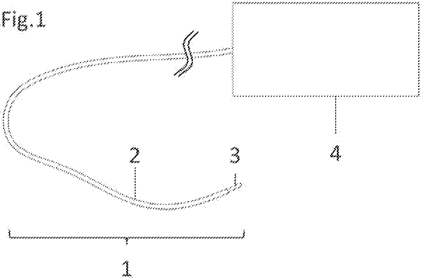

FIG. 1 in a schematic form, a connection line with a connected externally arranged consumer;

FIG. 2 a sectional representation through an example of a connection element;

FIG. 3 a sectional representation through a second example of a connection element; and

FIG. 4 a perspective representation of a third example of a connection element.

FIG. 1 shows a schematic representation of the arrangement for a temporary supply of a patient with a pacemaker as an external consumer 4. The electrically conductive connection element 1 consists of a line 2 and a polymer-free electrode region 3 and establishes a temporary electrically conductive connection to the electrical consumer 4 that is in particular an external pacemaker. In the case of application in accordance with the invention, the connection element 1 has been partially implanted in a patient so that at least the polymer-free electrode region is in contact with the heart tissue of the patient. The electrical consumer 4 is outside the body of the patient.

An example of a unipolar electrically conductive connection element 1 to be atrially anchored is shown in FIG. 2.

FIG. 2 shows a schematic sectional representation through an electrically conductive connection element 1 in a preferred embodiment for this purpose. This embodiment consists of metallic conductor strands 6 being wound around a core 5 that is formed from a polymer A that can be degraded in a short time. The conductor strands 6 are embedded in a first inner layer 7. The first inner layer 7 consists of the bioresorbable polymer A from which the core 5 is also formed. An outer layer 8 that is formed from a polymer B that is degradable more slowly in comparison with polymer A or from a polymer B that has a delayed start of the degradation in comparison with polymer A completely externally surrounds the first inner layer 7. The kind and design of the outer layer 8 of polymer B is selected such that the metallic conductor strand 6 is electrically insulated toward the surrounding body tissue for the intended time period of the connection to an eternal consumer 4 of a few days up to some weeks. The outer layer 8 of polymer B is subsequently degraded and exposes the first inner layer 7. The first inner layer 7, the conductor strands 6, and the core 5 dissolve in the body within a short time due to the higher degradation rate of polymer A and due to the preferred design of the conductor strands as very thin wires.

The metallic conductor strands 6 are formed from molybdenum wires having a circular cross-section and a diameter of 20 µm. The production of the wires takes place by wire drawing. 18 to 20 of these wires are wound around a central monofilament as a core 5 having a diameter of 100 µm. The central monofilament is formed from polylactide (PLA) having a molecular mass of 10,000 Da and reaches a high degradation rate under physiological conditions, designated as polymer A. This arrangement is coated with a first inner layer 7 having a thickness of 30 µm and composed of the same polymer A so that the molybdenum wires are completely surrounded by polymer A. This arrangement is subsequently coated with an outer layer 8 of 20 µm thickness and composed of polylactide having a molecular mass of 20,000 Da and a smaller degradation rate (polymer B). The outer layer 8 formed from polymer B surrounds the polymer A disposed thereunder and the conductor strands 6 over all sides and over the total length of the line 2. The electrode region 3 in which the metallic conductor strands 6 are exposed and that is provided for direct contact with the heart tissue is excluded from this.

The total outer diameter of this electrically conductive connection element 1 amounts to approximately 200 µm.

A premature contact of the conductor strands 6 with the surrounding tissue and an accompanying loss of function or a loss of the security of the connection element 1 is prevented by the outer layer 8 of polymer B that degrades over a longer time period.

As soon as the polymer B has degraded, the polymer A disposed thereunder swiftly dissolves. The very thin, now isolated molybdenum wires are likewise degraded very fast due to corrosion procedures.

A bipolar coaxially wound electrically conductive connection element 1 is shown in an embodiment in FIG. 3.

The metallic conductor strands 6 and 9 are formed from molybdenum wires having a circular cross-section and a diameter of 20 µm. The production of the wires takes place by wire drawing. 18 to 20 of these wires are wound around a central monofilament having a diameter of 50 µm and form the anodic conductor. The central monofilament is formed as a core 5 from polylactide (PLA) having a molecular mass of 10,000 Da and a high degradation rate under physiological conditions, designated as polymer A. This winding is coated with a first inner layer 7 having a thickness of 50 µm and composed of the same polymer A so that the molybdenum wires are completely surrounded by polymer A. A further 18 - 20 molybdenum wires having diameters of 20 µm are wound onto this arrangement as second metallic conductor strands 9 and form the cathodic conductor. A second inner layer 10 of polymer A of a thickness of 30 µm is applied to this second winding. This arrangement is subsequently coated with an outer layer 8 of 20 µm thickness and composed of polylactide having a molecular mass of 20,000 Da and a smaller degradation rate (polymer B). The total outer diameter of this electrically conductive connection element 1 amounts to approximately 250 µm.

A premature contact of the conductor strands 6 and 9 with the surrounding tissue and an accompanying loss of function or a loss of the security of the pacemaker line is prevented by the outer layer 8 of polymer B that degrades over a longer time period.

As soon as the polymer B has degraded, the polymer A disposed thereunder swiftly dissolves. The very thin, now isolated molybdenum wires are likewise degraded very fast due to corrosion procedures.

FIG. 4 shows a schematic representation of a connection element 1 having a modified polymer-free electrode region 3.

Reference numerals 5, 6, 7, and 8 refer to the same components of the connection line as are shown in FIG. 2. The coating 11 of the conductor strands 6 consists of a biocompatible, likewise bioresorbable and in this respect less oxidation sensitive metallic connection on a Mo or W base than the conductor strands 6 themselves. Possible application methods are inter alia a galvanic coating, chemical vapor deposition, plasma coating, and diffusion annealing. In a further embodiment, the coating 11 can be formed from bioresorbable, conductive oxides, nitrides, or carbides of the material of the conductor strands 6. These oxides, nitrides, or carbides can e.g. be obtained by a heat treatment of the electrical conductor strands 6 or also 9 by high temperature oxidation, nitration, or carbonization in an atmosphere rich in oxygen, nitrogen, or carbon.

What is claimed is:

1-14. (canceled)

15. An electrically conductive connection element for a temporary electrically conductive connection to an electrical consumer comprising an external pacemaker, implanted in the living tissue of a patient and consisting of at least one biocompatible and bioresorbable electrical conductor strand in the form of a single wire or of a multifilament formed from a plurality of wires, wherein the at least one electrical conductor strand is surrounded by at least one biocompatible and bioresorbable polymer or is embedded therein in a matrix formed by the polymer.

16. The connection element in accordance with claim 15, wherein the at least one electrical conductor strand is formed from molybdenum, tungsten, or an Mo or W based alloy.

17. The connection element in accordance with claim 15, wherein the at least one electrical conductor strand is formed from a Mo base alloy having at least 50 atomic weight % Mo and at least one of the alloy elements selected from W, Re, V, Nb, Ta, Hf, Mn and Fe; or in that the at least one electrical conductor strand is formed from a W base alloy having at least 50 atomic weight % W and at least one of the alloy elements selected from Mo, Re, V, Nb, Ta, Hf, Mn and Fe.

18. The connection element in accordance with claim 15, wherein the diameter of single wires with which the at least one electrical conductor strand is formed is in the range between 3 µm and 50 µm, preferably in the range between 10 µm and 30 µm.

19. The connection element in accordance with claim 15, wherein the diameter of single wires with which the at least one electrical conductor strand is formed is in the range between 10 µm and 30 µm.

20. The connection element in accordance with claim 15, wherein the at least one biocompatible and bioresorbable polymer is formed on the basis of polylactide, polyglycolide, poly(lactide-co-glycolide) or polycaprolactone.

21. The connection element in accordance with claim 15, wherein the at least one biocompatible and bioresorbable polymer is used as a homopolymer or as a copolymer or as a mixture of different ones of these polymers.

22. The connection element in accordance claim 15, wherein the at least one electrical conductor strand is surrounded by or embedded in a first inner layer formed by a polymer A and is surrounded by a second outer layer formed by a polymer B.

23. The connection element in accordance with claim 22, wherein the polymer B is degraded over a longer time period than the polymer A or has a delayed start of the degradation in comparison with polymer A.

24. The connection element in accordance with claim 22, wherein at least one electrical conductor strand is wound around a central core formed from polymer A and the first inner layer is formed on the surface of the core of polymer A provided with the electrical conductor strand and the first inner layer is in turn surrounded by an outer layer of polymer B.

25. The connection element in accordance with claim 22, wherein at least one further electrical conductor strand is wound on an outer surface of the first inner layer and at least one second inner layer having the polymer A is formed on the surface of the first inner layer having the at least one further electrical conductor strand and is in turn surrounded by the outer layer of the polymer B.

26. The connection element in accordance with claim 22, wherein a respective layer of polymer B is formed between the at least two inner layers that each surround at least one electrical conductor strand and are formed from polymer A.

27. The connection element in accordance with claim 15, wherein at least two electrical conductor strands having different functions are arranged coaxially or cordially with one another in at least one inner layer that is formed by polymer A.

28. The connection element in accordance with claim 27, wherein at least two electrical conductor strands are anodic and cathodic electrical conductors or are measurement, stimulation, and defibrillation probes.

29. The connection element in accordance with claim 15, wherein regions of the connection element that are provided for electrical contact with the living tissue are modified at their surfaces; and in that at least a portion of the at least one electrical conductor strand is kept free of polymer in these regions.

30. The connection element in accordance with claim 29, wherein at least the polymer-free regions of the at least one electrical conductor strand of an Mo or W base material are provided with a coating of a second Mo or W base material or from a bioresorbable conductive oxide, nitride, or carbide on a Mo or W base.
